(19) United States
(12) Patent Application Publication
(10) Pub. No.: US 2008/0091145 A1
(43) Pub. Date: Apr. 17, 2008

(54) Catheter assemblies with sized sheaths

(76) Inventor: Jamie Glen House, Colorado Springs, CO (US)

Correspondence Address: MOAZZAM & ASSOCIATES, LLC, 7601 LEWINSVILLE ROAD, SUITE 304, MCLEAN, VA, 22102, US

(21) Appl. No.: 11/546,238

(22) Filed: Oct. 12, 2006

(51) Int. Cl. A61M 5/00

(52) U.S. Cl. 604/171

(57) ABSTRACT

A grippable sheath for a catheter assembly is disclosed providing a sterile environment for a catheter. The grippable sheath may have a flattened diameter ranging from about 10 mm to about 50 mm. More specifically, the grippable sheath may have a flattened diameter ranging from about 15 mm to about 40 mm to provide improved usability and manipulability.

### BACKGROUND OF THE INVENTION

1. Field of the Invention

The present invention relates generally to catheter assemblies. More particularly, the present invention relates to catheter assemblies having sized sheaths.

2. Background of the Invention

It has become relatively commonplace for the occasional, intermittent or periodic catheterization of an individual's urinary bladder to be employed, as opposed to placement and maintenance of an indwelling catheter that continuously drains urine from the bladder. Short-term or repeated catheterization is appropriate, or even required, for many persons who are in a hospital setting, nursing home, doctor's office, rehabilitation facility, or in their own home. For example, a patient is sometimes catheterized in order to treat urinary retention, evacuate urine, or to obtain a sterile urine specimen from a patient.

The need for intermittent catheterization of an individual sometimes arises due to problems typically associated with long-term use of indwelling catheters such as infections, urethral damage, and bladder damage. Long-term use of an indwelling catheter is also a risk factor for bladder cancer. This is often the case for persons having a neurogenic urinary condition such as in a spinal cord injury, multiple sclerosis, stroke, trauma, or other brain injury. Other conditions that interfere with the individual's ability to voluntarily void the bladder may also arise post-surgically or as a result of benign prostatic hypertrophy or diabetes. Many of these affected individuals are capable of and would prefer to perform self-catheterization. For many, the level of risk and discomfort of repeated catheterizations carried out over the course of a day (at 3-6 hour intervals, for example) are offset by the accompanying convenience, privacy, and self-reliance that is achieved with self-catheterization. However, there are some major difficulties that arise in the current self-catheterization techniques which include the lack of satisfactory catheterization kits, the problem of maintaining the required level of sanitation during the procedure, difficulty in handling the catheter during insertion, and the difficulty of performing the procedure under conditions of restricted space and privacy.

In the assisted or non self-catheterizations presently employed in hospitals, it is common practice to use a catheterization tray. This tray typically includes a sterile drape, gloves, a conventional catheter, antiseptic solution, swabs, lubricant, forceps, an underpad, and a urine collection container. Assisted catheterization is usually performed with the patient in a supine position. The nurse typically grabs the patient's genitalia with one hand while using the other hand to insert the catheter into the patient's urethra. Thus, the nurse has only limited dexterity for manipulating the catheter in and around the patient's urethra. Also, maintaining a sterile field using “sterile technique” during the procedure can be a problem, and the “cath tray” procedure is impractical for use with some individuals having certain spastic and voluntary muscle disorders.

Many individuals with spinal cord injuries or other neurological diseases routinely perform intermittent catheterization several times a day using conventional catheters or kits and the “clean technique.” Clean technique means that the urethral area is initially swabbed with an antiseptic, and efforts are made to avoid contamination of the catheter during the procedure. The user's hands are not sterile and a sterile field is not maintained. Clean technique is used instead of sterile technique, generally, for two reasons. First, it is very difficult, if not impossible, for individuals who are performing self-catheterization to adhere strictly to sterile technique. Secondly, these individuals are required to catheterize themselves between 3 and 6 times a day, and the cost of a new sterile catheter and the accessories required to perform sterile catheterization becomes excessively expensive for some users. Sometimes an individual will reuse a “cleaned” catheter. As a result, the use of “clean technique” will many times result in contamination and subsequent infection of the urinary tract, causing significant morbidity and cost to the patient and society.

Thus, there is a need in the healthcare industry for a catheter assembly that enables all users, particularly those users with limited dexterity, to grip the catheter assembly and insert a contamination free catheter with decreased difficulty. Further, the catheter assembly should be easy to use and control, while having minimal parts that interfere with the catheterization process.

### SUMMARY OF THE INVENTION

The current techniques for catheterization are difficult to perform and are prone to contamination since it is very difficult for the user to maintain a sterile environment for the catheter during insertion. Also, the user may experience difficulty handling the catheter using the current catheterization processes. Thus, the present invention provides a grippable sheath having a defined diameter range in order to increase the efficiency and sterility of catheterization procedures.

In one exemplary embodiment, the present invention is a gripping device for a catheter. The device includes a sheath surrounding a catheter; and wherein the sheath is sized according to a diameter of the catheter and keeps the catheter in a sterile condition before and during insertion.

In another exemplary embodiment, the present invention is a gripping device for a catheter assembly. The device includes a sheath surrounding a catheter; and wherein the sheath is sized according to a diameter of the catheter and is attached at its proximal and distal ends to the catheter assembly so as to form leak-free seals preventing fluid communication between the lumen of the sheath and the external environment.

In another exemplary embodiment, the present invention is a gripping device for a catheter assembly. The device includes a sheath surrounding a catheter; and wherein the sheath has a diameter ranging from about 10 mm to about 50 mm.

In yet another exemplary embodiment, the present invention is a catheter assembly. The assembly includes a sheath surrounding a catheter; wherein the sheath is attached at its proximal and distal ends to the catheter assembly so as to form leak-free seals preventing fluid communication between the lumen of the sheath and the external environment; and wherein the sheath has a flattened diameter ranging from 15 mm to 40 mm.

As used herein and throughout this disclosure, and in order to understand the directional aspects of this invention, “proximal” refers to the section of the device that is closer to the patient's body (e.g., urethra) when the device is employed while “distal” refers to the section of the device that is farther away from the body, or closer to the back-end of the catheter assembly when the device is employed.

### BRIEF DESCRIPTION OF THE DRAWINGS

FIG. 1A shows a side view of a catheter assembly with a sheath according to an exemplary embodiment of the present invention.

FIG. 1B shows a side view of the catheter assembly in FIG. 1A after the sheath is pulled back from the catheter tip and body according to an exemplary embodiment of the present invention.

FIG. 2A shows a side view of a catheter assembly with a proximal guide portion and a sheath according to an exemplary embodiment of the present invention.

FIG. 2B shows a side view of a catheter assembly with a proximal guide portion and sheath after the sheath is pulled back from the catheter tip and body according to an exemplary embodiment of the present invention.

FIG. 3A shows an end view of the sheath and catheter from the catheter assemblies in FIG. 1A and FIG. 2A when the sheath is flattened out according to an exemplary embodiment of the present invention.

FIG. 3B shows a cross-section of the catheter assemblies in FIG. 1A and FIG. 2A while illustrating a diameter range for the sheath according to an exemplary embodiment of the present invention.

FIG. 4A shows a side view of a catheter assembly with a gas permeable sheath according to an exemplary embodiment of the present invention.

FIG. 4B shows a side view of the catheter assembly in FIG. 4A after the gas permeable sheath is pulled back from the catheter tip and body according to an exemplary embodiment of the present invention.

FIG. 5A shows a side view of a catheter assembly with a proximal guide portion and a gas permeable sheath according to an exemplary embodiment of the present invention.

FIG. 5B shows a side view of a catheter assembly with a proximal guide portion and a gas permeable sheath after the sheath is pulled back from the catheter tip and body according to an exemplary embodiment of the present invention.

FIG. 6A shows an end view of the sheath and catheter from the catheter assemblies in FIG. 4A and FIG. 5A when the sheath is flattened out according to an exemplary embodiment of the present invention.

FIG. 6B shows a cross-section of the catheter assemblies in FIG. 4A and FIG. 5A while illustrating a diameter range for the gas permeable sheath according to an exemplary embodiment of the present invention.

### DETAILED DESCRIPTION OF THE INVENTION

The present invention provides for catheter and catheter assemblies with easy-gripping sheaths having a diameter specifically defined so that such catheter and catheter assemblies are contamination free and are more readily manipulated in and around the body. In particular embodiments and examples presented herein, such catheters are described with respect to urinary catheterization, but it should be noted that such sheaths according to the present invention are not limited to urinary catheters alone but may be applicable to any catheter and catheter assembly that could benefit from the use of such sheaths. Furthermore, the present invention allows for more efficient and secure handling of catheter and catheter assemblies to decrease the time required for and increase the safety and reliability of catheterization and associated procedures.

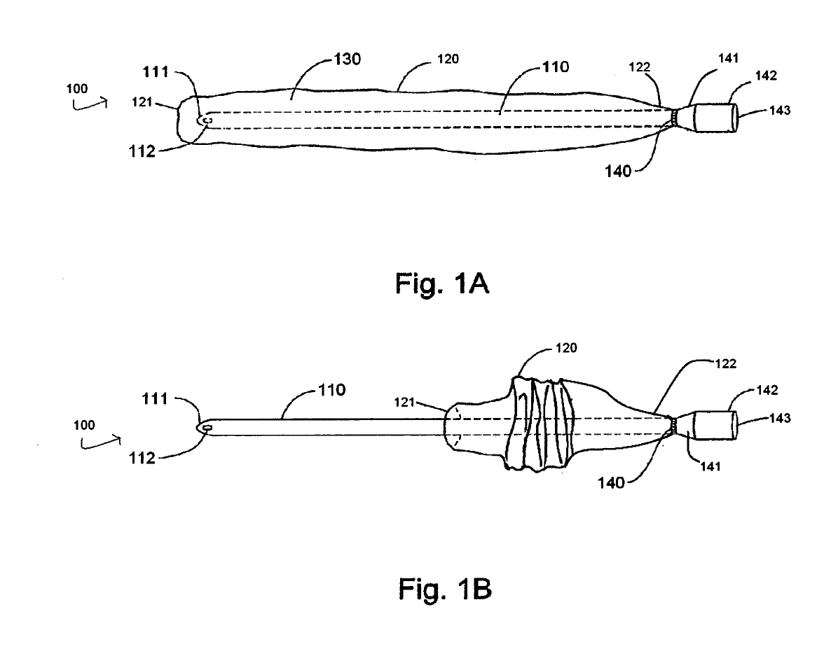

An exemplary embodiment of the present invention as used in catheter assembly 100 is shown in FIG. 1A. Assembly 100 includes a catheter 110 with a fluid receiving aperture 112, a sheath 120, lubricant 130, distal terminus connector 141, distal terminus 142, and a urine outlet 143. The sheath 120 may be composed of any material such that it may be easily grasped and that it may function to maintain a sterile internal environment for the catheter 110. The sheath 120 may also provide a reservoir for lubricant 130 since it may be composed of a liquid-impermeable material. The sheath 120 may be comprised of artificial or naturally occurring non-degradable biocompatible polymer compounds such that the materials used serve the function of providing an external gripping surface and an internal sterile environment. Such compounds can include, but are not limited to, polyester based biocompatible polymers, nylon-based biocompatible polymers, latex based biocompatible polymers, Teflon, polytetrafluoroethylene (PTFE) polymers, polyvinyl chloride (PVC) polymers, silicone polymers, polyurethane polymers, silicone polyurethane polymers, ethylene-vinyl acetate copolymers, polyethylene polymers, and thermoplastic polymers.

During catheterization, the sheath 120 may be grasped with one hand while the other hand may be used to advance the catheter 110 through the proximal end 121 of the sheath 120 and into the patient's urethra. As the catheter 110 is advanced into the patient's urethra, the sheath 120 may be guided down the body of catheter 110 toward the distal terminus 142 as shown in FIG. 1B. The sheath 120 may collect as shown in FIG. 1B in an accordion-like fashion in order to allow for a more efficient and easier catheterization. Also, the sheath 120 may be of such a diameter so as to maximize user grippability while minimizing user difficulty in grasping and guiding the catheter 110. Thus, the proximal end 121 of the sheath 120 may be of a relative diameter as depicted in FIG. 1A or FIG. 1B or it may be tighter or looser around the catheter 110 such that the sheath 120 is able to easily be manipulated into a compressed accordion-like shape such that interference and/or resistance to the catheter insertion is prevented and/or effectively minimized. In order to achieve this functionality, the sheath 120 may sized such that a substantially flattened diameter falls within the range of about 10 mm to about 50 mm. More specifically, the sheath 120 may have a flattened diameter ranging from 15 mm to 40 mm. The catheter 110 may have a circular diameter ranging from 2 mm to 8 mm. With these sheath diameter ranges, the usability and functionality of assembly 100 may be maximized to allow the sheath 120 to roll up and collect with the least difficulty at the distal end of assembly 100 as shown in FIG. 1B during catheter use. The flattened diameter for the sheath 120 given above is intended to provide a most cost efficient size for manufacturing feasibility while providing a large enough surface for the user to effectively grip and therefore handle the catheter 110 while it is inside the sheath 120. It must be noted that the diameter ranges as specified above for the sheath 120 were found to provide an optimal usability. For example, a sheath length less than 10 mm may result in the sheath 120 experiencing too much resistance to catheter insertion thereby making it difficult to slide over the catheter 110. This increased resistance occurs when the sheath becomes smaller (e.g., <15 mm) because as the sheath bunches, it has no room to go except to press down further into the catheter. Since this occurs 360 degrees around the catheter, it closes in on the catheter and grips it, preventing it from sliding within the sheath. Conversely, with a sheath greater than 50 mm, this may result in the sheath 120 being too cumbersome to manipulate.

The sheath 120 may collect just proximal to the distal connector 141 when the catheter 110 is fully inserted into the patient's bladder. This allows the user optimal usability of assembly 100 since the sheath 120 can neatly collect at the distal end 122 thereby avoiding interference with the catheter 110 during insertion. After catheterization, the user may unfold the accordion-shaped sheath 120 shown in FIG. 1B and guide the proximal end 121 of sheath 120 back towards the proximal tip 111 of catheter 110 during catheter removal to further prevent contamination.

The distal end 122 of sheath 120 may be attached to distal connector 141 via attachment 140 in such a way so as to provide a leak-free seal in order to store the lubricant 130 inside the sheath 120. Such an attachment method may include but is not limited to heat sealing, heath shrinking, adhesive collaring, etc. The distal tip of catheter 110 may be situated inside the distal terminus 142 in order for the collected urine to drain out through the urine outlet 143 and into a collection receptacle (not shown). The amount of lubricant 130 may vary and may be of such an amount so as to lubricate at least the insertable portion of catheter 110. The length of the sheath 120 may be at least equivalent to the length of the insertable portion of catheter 110 which may be used to enter the patient's bladder.

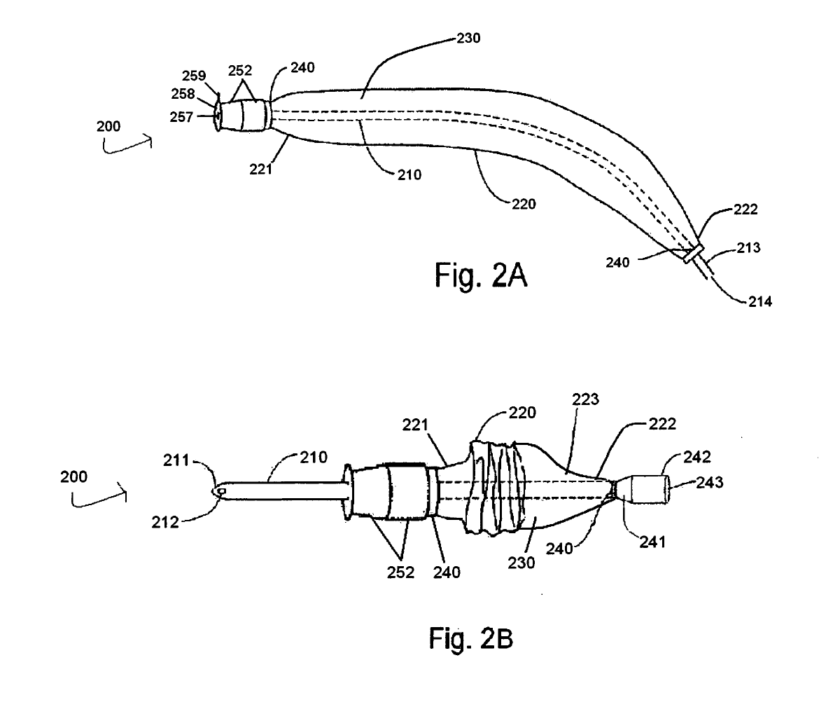

Another exemplary embodiment of the present invention as used in catheter assembly 200 is shown in FIG. 2A. Assembly 200 includes proximal guide portion 252, catheter 210, sheath 220, and lubricant 230. The sheath 220 may function as similarly described above. This exemplary embodiment of the present invention incorporates a proximal guide portion 252 which may be used to both stabilize assembly 200 and guide catheter 210 into the patient's urethra. The proximal guide portion 252 may be frustoconical in shape with a rectangularly shaped distal piece connected to a conically shaped proximal piece as shown in FIG. 2A. An apertured center 257 of proximal phalange 258 on the proximal guide portion 252 may function as a guiding channel for the catheter 210 to pass. The proximal phalange 258 may be the same size as but is not limited to the size of a nickel. The proximal guide portion 252 may house additional lubricant in order to facilitate catheter insertion. The proximal phalange 258 may possess a seal or covering (not shown) to prevent any lubricant 230 from the sheath and the proximal guide portion 252 from prematurely leaking into the external environment. The seal or covering may be removed by grasping a seal tab 259 and ripping the seal or covering off of the proximal phalange 258. Various exemplary embodiments of the proximal guide portion 252 as used herein may be found in U.S. Pat. No. 6,090,075, entitled “Disposable urinary catheterization assembly”, issued to House, which is incorporated by reference herein in its entirety.

Both the proximal end 221 and the distal end 222 of sheath 220 may be connected to the catheter 210 via attachment site 240. The proximal end 221 of the sheath 220 may be of a relative diameter as depicted in FIG. 2A or FIG. 2B or it may be tighter or looser around the catheter 210 such that the sheath 220 is able to easily be manipulated into a compressed accordion-like shape such that interference and/or resistance to the catheter insertion is prevented and/or effectively minimized. Attachment site 240 may be achieved using any of the methods as described above for FIG. 1 so as to provide a leak-free seal. Alternatively, the distal attachment site 240 may not be bound to catheter 210 and would therefore be free to translate and slide over the catheter 210 as is shown in FIG. 2A. Thus, the attachment site 240 may have a circular diameter slightly larger than the circular diameter of catheter 210 in order to allow it to slide over the catheter 210. The distal end 213 of catheter 210 may extend past the distal attachment site 240 as shown in FIG. 2A so that the collected urine may drain through outlet 214.

In order to achieve optimal user grippability and manipulability of the catheter 210, the sheath 220 may range from 10 mm to about 50 mm in flattened diameter while the catheter 210 may range from 2 mm to 8 mm in circular diameter. More specifically, the flattened diameter of sheath 220 may range from about 15 mm to about 40 mm. The sheath may be composed of the same materials as described above as well.

During catheter insertion, the user may grasp the proximal guide portion 252 with one hand while grasping the catheter 210 through the sheath 220 with the other hand. As the catheter is guided through the apertured center 257 and into the patient's urethra, the sheath 220 may or may not bunch up and form an accordion-like shape depending on whether or not the attachment site 240 forms a leak-free seal between the inside of the sheath 220 and the external environment. In either case, the sheath 220 may be about 10 mm to about 50 mm in flattened diameter so as to allow for optimal gripping and handling power. More specifically, the flattened diameter of sheath 220 may range from about 15 mm to about 40 mm to further facilitate handling of assembly 200. If the leak-free seal is formed at attachment site 240, then the specified diameter ranges may further serve to prevent an excess of air build-up within the sheath 220 which would act to resist catheter insertion.

Another exemplary embodiment of the present invention is shown in FIG. 2B. In FIG. 2B, a variation of assembly 200 is shown which incorporates a distal connector 241, a distal terminus 242, and a urine outlet 243. The exemplary embodiment in FIG. 2B includes the same component elements as the exemplary embodiment illustrated in FIG. 2A (e.g., catheter 210, sheath 220, etc) and is subject to the same limitations described in the exemplary embodiment shown in FIG. 2A, but this exemplary embodiment further incorporates the distal connector 241, distal terminus 242, and urine outlet 243. The proximal end 221 and the distal end 222 of sheath 220 may be sealed via attachment sites 240 so as to provide a leak-free and contamination free environment inside the sheath 220. The method used for the attachment may include the methods as described above so long as a liquid impermeable barrier is created between the inside and outside of sheath 220.

The sheath 220 may be of such a diameter so as to maximize user grippability while minimizing user difficulty in grasping and guiding the catheter 220. In order to achieve this functionability, the sheath 220 may be of a flattened diameter falling within the range of about 10 mm to about 50 mm. Such a range may allow the sheath the ability to roll up and collect with the least difficulty at the distal end of assembly 200 as shown in FIG. 2B during catheter use. More specifically, the sheath 220 may have a flattened diameter ranging from about 15 mm to about 40 mm in order to provide maximal gripping ability while avoiding the clumsiness of excess sheath material.

As the catheter 210 is advanced into the patient's urethra, the sheath 220 may be guided down the body of catheter 210 toward the distal terminus 242 as shown in FIG. 2B. The sheath 220 may collect as shown in FIG. 2B in an accordion-like fashion in order to allow for a more efficient and easier catheterization. During catheterization, a slight increase in air pressure within the sheath 220 at specifically location 223 may be observed since there is no gas communication between the inside and the outside of the assembly. However, using the 10 mm to 50 mm range as described, or more specifically the 15 mm to 40 mm range, may help avoid an excessive buildup air pressure which could impede the collecting of the sheath 220 at the distal end 222 of assembly 200. Also, in combination with said specified diameter range, there could be an amount of lubricant 230 just enough to coat and lubricate the insertable portion of catheter 210 but not so much so as to take up too much volume within the sheath 220 which could lead to an increase in air pressure as well. The 10 mm to 50 mm range, and more specifically the 15 to 40 mm range, as presented herein may ensure the best balance between cost efficiency and user manipulability.

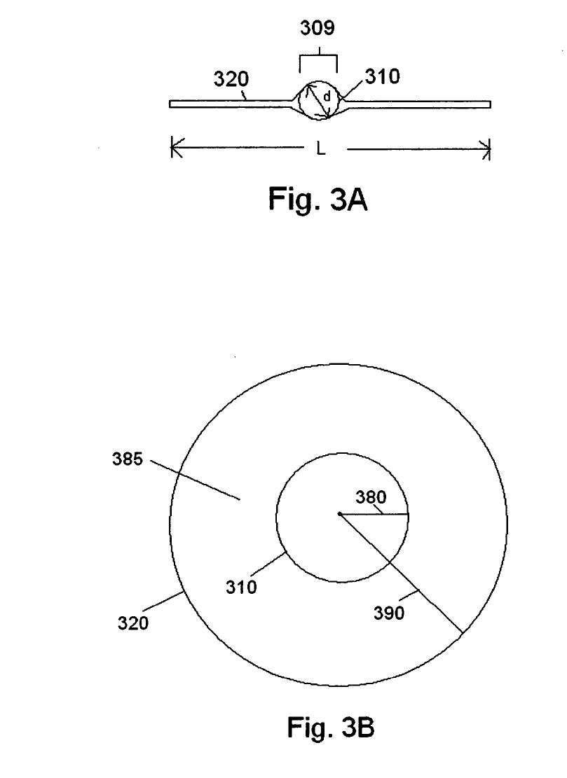

FIG. 3A shows a side view of what the sheath 320 and catheter 310 would look like in order to measure the flattened diameter of sheath 320. Although the exact flattened diameter of the sheath may not be obtained due to the presence of the catheter 310, the measurement may nonetheless be recorded ignoring the slight unflattened part 309 for sake of simplicity. According to an exemplary embodiment of the present invention, d may range from 2 mm to 8 mm and L may range from about 10 mm to about 50 mm. Thus, a d/L ratio, which signifies a catheter diameter to flattened sheath diameter ratio, ranges from about 1:5 to about 1:25 for a d of 2 mm, and ranges from 4:5 to 1:6 for a d of 8 mm. More specifically, L may range from about 15 mm to about 40 mm. Thus, a d/L ratio ranges from about 1:7.5 to about 1:20 for a d of 2 mm, and ranges from about 1:2 to about 1:5 for a d of 8 mm.

FIG. 3B shows a cross-section of the catheter assemblies as described above in FIG. 1A and FIG. 2A. FIG. 3B includes a catheter 310, a sheath 320, and a lumen 385. According to one exemplary embodiment of the present invention, the circular radius 380 of the catheter 310 may be equal to about 1.5 mm if the circular diameter of the catheter 310 is about 3 mm (e.g., falling within the range of 2 mm to 8 mm as specified above) as described according to an exemplary embodiment of the present invention. The circular radius 390 of the sheath 320 may therefore range from about 3.18 mm to about 15.92 mm according to an exemplary embodiment of the present invention. These numbers coincide with a flattened diameter range for the sheath 320 of about 10 mm to about 50 mm. Thus, given the broader range of about 10 mm to about 50 mm of the circular diameter of sheath 320, the ratio of sheath flattened diameter to catheter circular diameter may be as great as about 5:1. More specifically, with a flattened diameter range of 15 mm to 40 mm according to an exemplary embodiment of the present invention, the radius 390 of the sheath 320 may range from 4.77 mm to 12.74 mm. Thus, given the more narrow range of 15 mm to 40 mm of the circular diameter of sheath 320, the ratio of sheath flattened diameter to catheter circular diameter may be as great as roughly 4:1.

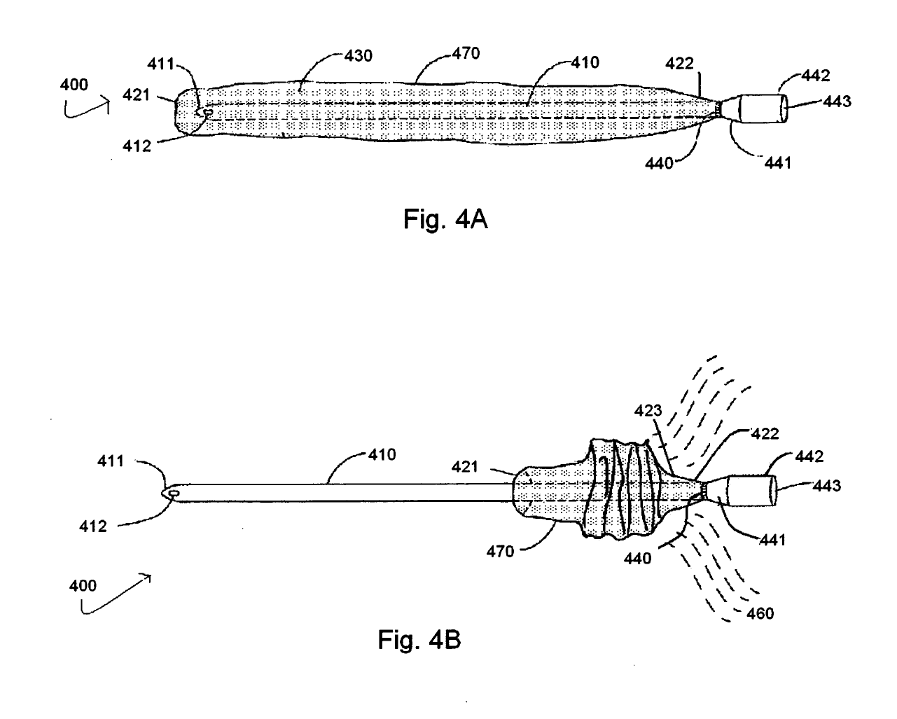

Another exemplary embodiment of the present invention is catheter assembly 400 and is shown in FIG. 4A. Assembly 400 as show in FIG. 4A corresponds with the assembly in FIG. 1A in the sense that the corresponding labeled components possess the same characteristics and are subject to the same parameters (e.g., catheter 110 corresponds to catheter 410, etc.). However, the sheath 470 differs from the sheath in FIG. 1A in that sheath 470 is composed of a liquid-impermeable, gas-permeable material such that air may flow through the sheath material thereby providing a means for gas exchange between the inside and outside of the sheath 470. Reference is herein made to copending application ______, filed by House, entitled “Catheter Assembly Having protective Sheath,” to further describe the gas-permeable but liquid-impermeable material.

FIG. 4B shows the collecting of the proximal end 422 of the sheath 470 during catheter insertion and corresponds with FIG. 1B. In combination with the gas-permeable material used for sheath 470, a diameter range of about 10 mm to about 50 mm for the sheath 470 may allow for optimal handling of assembly 400 since an increase in air pressure within the sheath 470 may be avoided by both the sheath material and the specified sheath diameter. More specifically, the sheath diameter may range from about 15 mm to about 40 mm in order to minimize any excess air build up inside the sheath and in particular at sheath location 423. The direction of air flow from within the sheath 470 to the external environment is shown in FIG. 4B as well via arrows 460. The accordion-like collection of the sheath 470 is also shown in FIG. 4B near distal end 422 according to an exemplary embodiment of the present invention.

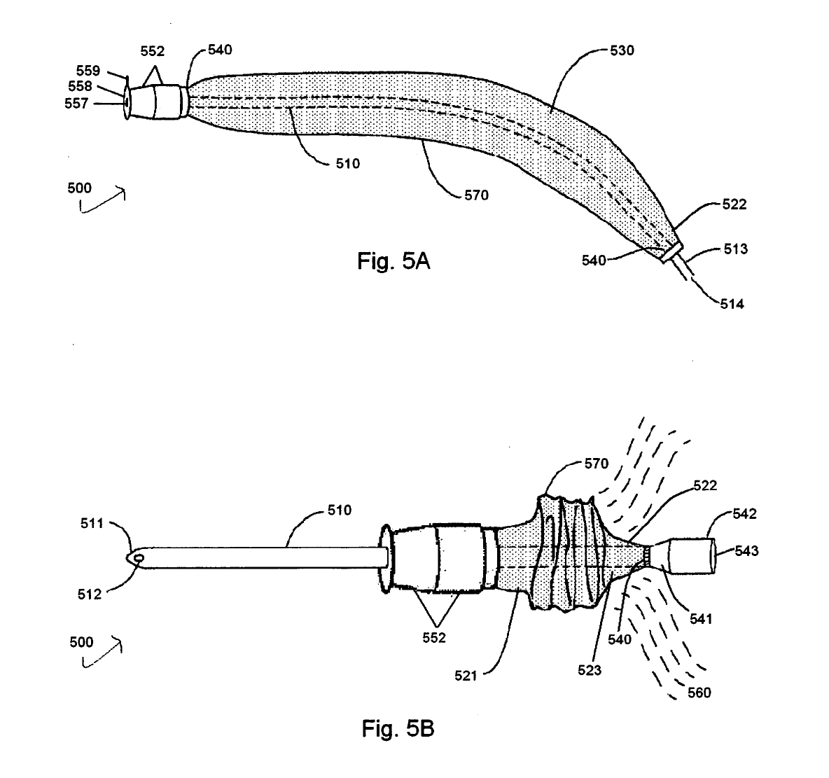

Another exemplary embodiment of the present invention is catheter assembly 500 and is shown in FIG. 5A. Assembly 500 as show in FIG. 5A corresponds with the assembly in FIG. 2A in the sense that the corresponding labeled components possess the same characteristics and are subject to the same parameters (e.g., catheter 210 corresponds to catheter 510, etc.). However, the sheath 570 differs from the sheath in FIG. 2A in that sheath 570 is composed of a liquid-impermeable, gas-permeable material such that air may flow through the sheath material.

FIG. 5B shows the collecting of the proximal end 522 of the sheath 570 during catheter insertion and corresponds with FIG. 2B. In combination with the gas-permeable material used for sheath 570, a diameter range of about 10 mm to about 50 mm for the sheath 570 may allow for optimal handling of assembly 500 since an increase in air pressure within the sheath 570 may be avoided by both the sheath material and the specified sheath diameter. More specifically, the sheath diameter may range from 15 mm to 40 mm in order to minimize any excess air build up inside the sheath and in particular at sheath location 523. The direction of air flow from within the sheath 570 to the external environment is shown in FIG. 5B as well via arrows 560. The accordion-like collection of the sheath 570 is also shown in FIG. 5B near distal end 522 according to an exemplary embodiment of the present invention.

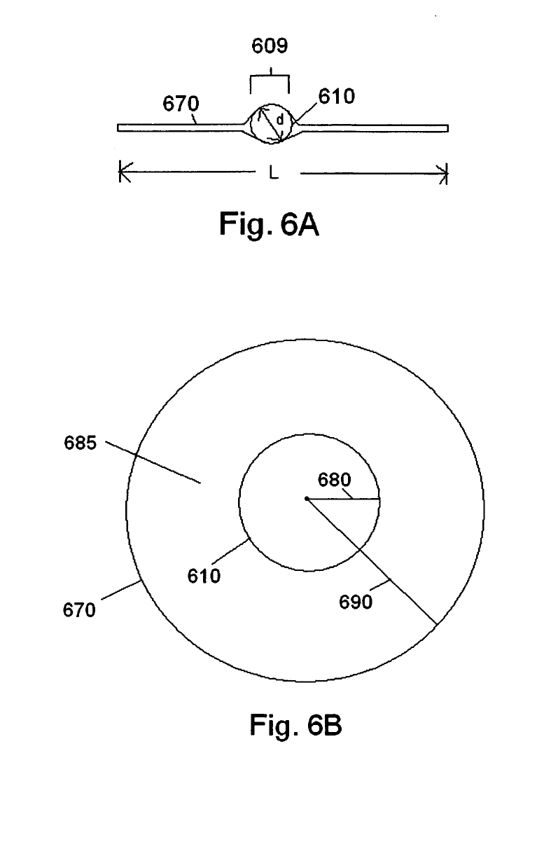

FIG. 6A shows a side view of what the sheath 670 and catheter 610 would look like in order to measure the flattened diameter of sheath 670. Although the exact flattened diameter of the sheath may not be obtained due to the presence of the catheter 610 inside, measurement may nonetheless be recorded ignoring the slight unflattened portion 609 for the sake of simplicity. According to an exemplary embodiment of the present invention, d may range from 2 mm to 8 mm and L may range from about 10 mm to about 50 mm. Thus, a d/L ratio, which signifies a catheter diameter to flattened sheath diameter ratio, ranges from about 1:5 to about 1:25 for a d of 2 mm, and ranges from 4:5 to 1:6 for a d of 8 mm. More specifically, L may range from about 15 mm to about 40 mm. Thus, a d/L ratio ranges from about 1:7.5 to about 1:20 for a d of 2 mm, and ranges from about 1:2 to about 1:5 for a d of 8 mm.

FIG. 6B shows a cross-section of the catheter assemblies as described above in FIG. 4A and FIG. 5A. FIG. 6B includes a catheter 610, a sheath 670, and a lumen 685. According to one exemplary embodiment of the present invention, the circular radius 680 of the catheter 610 may be equal to about 1.5 mm if the circular diameter of the catheter 610 is about 3 mm (e.g., falling within the range of 2 mm to 8 mm as specified above) as described according to an exemplary embodiment of the present invention. The circular radius 690 of the sheath 670 may therefore range from about 3.18 mm to about 15.92 mm according to an exemplary embodiment of the present invention. These numbers coincide with a flattened diameter range for the sheath 670 of about 10 mm to about 50 mm. Thus, given the broadest range of 10 mm to 50 mm of the circular diameter of sheath 670, the ratio of sheath flattened diameter to catheter circular diameter may be as great as about 5:1. More specifically, with a flattened diameter range of about 15 mm to about 40 mm according to an exemplary embodiment of the present invention, the radius 690 of the sheath 670 may range from about 4.77 mm to about 12.74 mm. These numbers coincide with a flattened diameter range for the sheath 670 of about 15 mm to about 40 mm. Thus, given the more narrow range of about 15 mm to about 40 mm of the circular diameter of sheath 670, the ratio of sheath flattened diameter to catheter circular diameter may be as great as about 4:1.

If not specified otherwise in the description above, all of the materials used for the present invention may be comprised of artificial or naturally occurring non-degradable biocompatible polymer compounds such that the materials used for the present invention serve the functions delineated in this application. Such compounds can include, but are not limited to, polyester based biocompatible polymers, nylon-based biocompatible polymers, latex based biocompatible polymers, Teflon, polytetrafluoroethylene (PTFE) polymers, polyvinyl chloride (PVC) polymers, silicone polymers, polyurethane polymers, silicone polyurethane polymers, ethylene-vinyl acetate copolymers, polyethylene polymers, and thermoplastic polymers. The catheters themselves may also be composed of a hydrophilic compound formed from, but not limited to, the reaction of an epoxy containing polyvinyl pyrrolidone with a polyamino compound. The lubricating material includes but is not limited to an aqueous solution or a hydrogel formed as the reaction product of a siloxane containing macromer, one hydrophilic polymer, and one compatibilizing component. The lubricating material also may be composed of a biocompatible antibacterial agent such as a 4-quinolone agent to prevent contamination.

The manufacturing methods that can be employed for the present invention include, but are not limited to, conventional techniques used in the industry to produce similar function products, as known by a person having ordinary skill in the catheter art.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A gripping device for a catheter, the device comprising:
a sheath surrounding a catheter; and
wherein the sheath is sized according to a diameter of the catheter and keeps the catheter in a sterile condition before and during insertion.

2. The device of claim 1, wherein the sheath has a flattened diameter ranging from about 10 mm to about 50 mm.

3. The device of claim 2, wherein a catheter diameter to flattened sheath diameter ratio ranges from about 1:3 to about 1:17.

4. The device of claim 2, wherein the sheath has a flattened diameter ranging from about 15 mm to about 40 mm.

5. The device of claim 4, wherein a catheter diameter to flattened sheath diameter ratio ranges from about 1:5 to about 1:13.

6. A gripping device for a catheter assembly, the device comprising:
a sheath surrounding a catheter; and
wherein the sheath is sized according to a diameter of the catheter and is attached at its proximal and distal ends to the catheter assembly so as to form leak-free seals preventing fluid communication between the lumen of the sheath and the external environment.

7. The device of claim 6, wherein the sheath has a flattened diameter ranging from about 10 mm to about 50 mm.

8. The device of claim 7, wherein a catheter diameter to flattened sheath diameter ratio ranges from about 1:3 to about 1:17.

9. The device of claim 7, wherein the sheath has a flattened diameter ranging from about 15 mm to about 40 mm.

10. The device of claim 9, wherein a catheter diameter to flattened sheath diameter ratio ranges from about 1:5 to about 1:13.

11. A gripping device for a catheter assembly, the device comprising:
a sheath surrounding a catheter; and
wherein the sheath has a diameter ranging from about 10 mm to about 50 mm.

12. The device of claim 11, wherein a catheter diameter to a flattened sheath diameter ratio ranges from about 1:3 to about 1:17.

13. The device of claim 11, wherein the sheath has a flattened diameter ranging from about 15 mm to about 40 mm.

14. The device of claim 13, wherein a catheter diameter to flattened sheath diameter ratio ranges from about 1:5 to about 1:13.

15. The device of claim 11, wherein the sheath is permeable.

16. A catheter assembly, the assembly comprising:
a sheath surrounding a catheter;
wherein the sheath is attached at its proximal and distal ends to the catheter assembly so as to form leak-free seals preventing fluid communication between the lumen of the sheath and the external environment; and
wherein the sheath has a flattened diameter ranging from 15 mm to 40 mm.

17. The assembly of claim 16, wherein a catheter diameter to a flattened sheath diameter ratio ranges from about 1:3 to about 1:17.

18. The device of claim 16, wherein the sheath has a flattened diameter ranging from about 15 mm to about 40 mm.

19. The device of claim 18, wherein a catheter diameter to flattened sheath diameter ratio ranges from about 1:5 to about 1:13.

20. The device of claim 16, wherein the sheath is permeable.
